(19) United States
(12) Patent Application Publication
(10) Pub. No.: US 2010/0120047 A1
(43) Pub. Date: May 13, 2010

(54) PURIFICATION OF TARGET CELLS FROM COMPLEX BIOLOGICAL FLUIDS

(75) Inventor: Roger Allyn Forsyth, San Diego, CA (US)

Correspondence Address: James P. Broder;Roeder & Broder LLP, 9915 Mira Mesa Blvd. Suite 300, San Diego, CA, 92131, US

(73) Assignee: GHC Technologies, Inc.

(21) Appl. No.: 12/613,907

(22) Filed: Nov. 6, 2009

Related U.S. Application Data
(60) Provisional application No. 61/112,516, filed on Nov. 7, 2008.

(51) Int. Cl. C12Q 1/68; C12Q 1/02

(52) U.S. Cl. 435/6; 435/29

(57) ABSTRACT

A method for conducting a molecular analysis on target cells of a complex biological fluid can include the steps of reducing the complexity by selectively depleting at least a portion of non-target cells from the biological fluid, labeling target cells or non-target cells for identification, isolating target cells from non-target cells based on (i) a size, or (ii) a label, of the target cells or the non-target cells using a microfluidic sorting apparatus, and molecularly analyzing the target cells. Reducing can include separating the non-target cells from the biological fluid using a Magnetic Activated Cell Sorting apparatus or using magnetic beads that include a plurality of different antibodies including one or more of CD45, CD16 and 235a. Labeling can include using a label selected from fluorescent, colorimetric, magnetic and biochemical labels. The biological fluid can be selected from blood sputum, peritoneal fluid, tissue cell suspension, fine needle aspirates, fecal matter and cerebral spinal fluid. The target cells can be selected from tumor cells, fetal cells, stem cells, endothelial cells, myocardial cells and lymphocytes. The step of molecularly analyzing the target cells can include one of RNA analysis, antigen expression and mutation profiling.

### REFERENCE TO RELATED APPLICATION

This Application claims domestic priority on U.S. Provisional Application Ser. No. 61/112,516, filed on Nov. 7, 2008. The contents of U.S. Provisional Application Ser. Nos. 61/112,516 are incorporated herein by reference to the extent permitted.

### BACKGROUND

For years, many researchers have attempted to perform molecular analyses on rare cells (also referred to herein as “target cells”) in various biological fluids (e.g., blood) that also contain assorted non-target cells. For example, one conventional system has had a certain degree of success at enumeration of circulating tumor cells (CTCs) as one type of target cell. However, this system has encountered extreme difficulty in evaluating a mutation status or analyzing mRNA transcipt levels of the target cells primarily because the background level of the non-target cells is too high, creating a target cell purity that is too low. For instance, the ratio of target cells to non-target cells can be 1 to 100, 1 to 1,000, or even lower.

Additionally, attempts at enabling selective transcript analysis have not been wholly successful. Some of these techniques avoid the problem of background non-target cells by analyzing only the mRNA transcripts which are uniquely expressed in the target cells, and which are not expressed in the non-target cells. In other words, with these types of systems, the apparent purity of the measured mRNA transcripts is essentially 100% because the non-target cells do not express mRNA transcripts. One such system analyzes unique target mRNA transcripts from enriched blood samples but is grossly restricted to just a few mRNA measurements which meet this expression criterion.

### SUMMARY

The present invention is directed toward a method for conducting a molecular analysis on target cells of a complex biological fluid that includes target cells and non-target cells, the method comprising the steps of reducing the complexity of the biological fluid by selectively depleting at least a portion of the non-target cells from the biological fluid, labeling the target cells or the non-target cells for identification, isolating the target cells from the non-target cells based on one of (i) a size, or (ii) a label, of the target cells or the non-target cells using a microfluidic sorting apparatus, and molecularly analyzing the target cells.

In one embodiment: step of reducing includes separating the non-target cells from the biological fluid using a Magnetic Activated Cell Sorting apparatus. In another embodiment, the step of separating includes using magnetic beads that include a plurality of different antibodies. The antibodies can include one or more of CD45, CD16 and 235a Further, the step of reducing can include lysing at least some of the non-target cells. The step of lysing can include using an ammonium chloride solution. The step of reducing can also or alternatively include filtration of at least some of the non-target cells.

In certain embodiments, the step of labeling can include labeling both the target cells and the non-target cells. In another embodiment, the step of labeling can include using a label selected from the group consisting of fluorescent, colorimetric, magnetic and biochemical labels. Additionally, or in the alternative, the microfluidic sorting apparatus can include a microfluidic Fluorescence Activated Cell Sorting apparatus.

In one embodiment, the step of isolating includes purifying the target cells based on a label of the target cells and the non-target cells. The step of isolating can include purifying the target cells based on the size and the label of either the target cells or the non-target cells. Further, in one embodiment, the number of target and non-target cells in the biological fluid can be less than approximately 1×10¹¹ cells in a fluid volume of less than approximately 100 mls.

In another embodiment, the step of isolating can include recovering the target cells in a volume of at least approximately 5 μl and less than approximately 1 ml. Further, the step of isolating can include recovering the target cells in a volume of at least approximately 10 μl and less than approximately 0.1 ml. In one embodiment, the biological fluid is selected from the group consisting of blood sputum, peritoneal fluid, tissue cell suspension, fine needle aspirates, fecal matter and cerebral spinal fluid. In another embodiment, the step of isolating yields a purity of at least approximately 60%.

Further, the target cells can be selected from the group consisting of tumor cells, fetal cells, stem cells, endothelial cells, myocardial cells and lymphocytes. In some embodiments, the duration of the method is less than approximately 6 hours. In certain embodiments, the step of isolating includes retention of the target cells being at least 80%. In another embodiment, the step of molecularly analyzing the target cells can include one of RNA analysis, antigen expression and mutation profiling.

### BRIEF DESCRIPTION OF THE DRAWINGS

The invention, together with further advantages thereof, may best be understood by reference to the following description taken in conjunction with the accompanying drawings, taken in conjunction with the accompanying description, in which similar reference characters refer to similar parts, and in which:

FIG. 1 is a flow chart illustrating one embodiment of a method for conducting a molecular analysis on target cells of a complex biological fluid that includes target cells and non-target cells;

FIG. 2 is a flow chart illustrating one embodiment of a method for reducing complexity of the complex biological fluid by selectively depleting at least a portion of the non-target cells;

FIG. 3A is a graph of Cytokeratin PE as a function of CD45 PE-Cy5 for whole blood spiked-in with 100 prostate cancer cells (target cells) treated with taxol, illustrating a relatively high rate of recovery of target cells;

FIG. 3B is a graph of phosphorylated histone H3-A488 as a function of peak intensity (forward side scatter—FSC) for the recovered target cells in FIG. 3A;

FIG. 4A is a graph of Ct values from a qRT-PCR analysis of cell transcripts of a biological fluid that includes approximately 50 spiked-in target cells compared to the Ct values of the same transcripts when analyzed from target cells sorted from whole blood;

FIG. 4B is a graph of Ct values from a qRT-PCR analysis of cell transcripts of blood cells in the biological fluid described relative to FIG. 3A following magnetic depletion, compared to the Ct values of the same transcripts when analyzed from target cells sorted from whole blood;

FIG. 4C is a graph of Ct values from qRT-PCR analysis of cell transcripts of blood cells in the biological fluid described relative to FIG. 3B following sorting by a microfluidic Fluorescence Activated Cell Sorting apparatus, compared to the Ct values of the same transcripts when analyzed from target cells sorted from whole blood; and

FIG. 5 is a table illustrating how molecular analysis including accurate mutation detection in wild type and heterozygous strains is enabled from a few cells isolated from whole blood by the methods that follow the steps of the present invention.

### DESCRIPTION

The methods provided herein allow an increased purification of rare cells (also referred to herein as “target cells”) from complex biological fluids that also include non-rare cells (also referred to herein as “non-target cells”). The process uniquely utilizes techniques that yield unexpected results, i.e. rapid, efficient recovery of purified rare cells which have utility in a variety of assays. As provided herein, the duration of the described processes is decreased from conventional processes, while enabling expedited diagnoses, enhancing viability and reducing and/or eliminating changes to the biology of cells. In certain embodiments, relatively large volumes such as 100 mLs of blood can be reduced to 5 μl or less of a substantially pure population of target cells which can be initially present at extremely low levels, such as only 1 cell/mL or less. In one embodiment, the purity approaches or even achieve approximately 100% with recoveries routinely exceeding approximately 80%. The collection of essentially pure target cells has enabled a range of analyses, biological insights and treatment modalities that were previously impractical, if not impossible to attain.

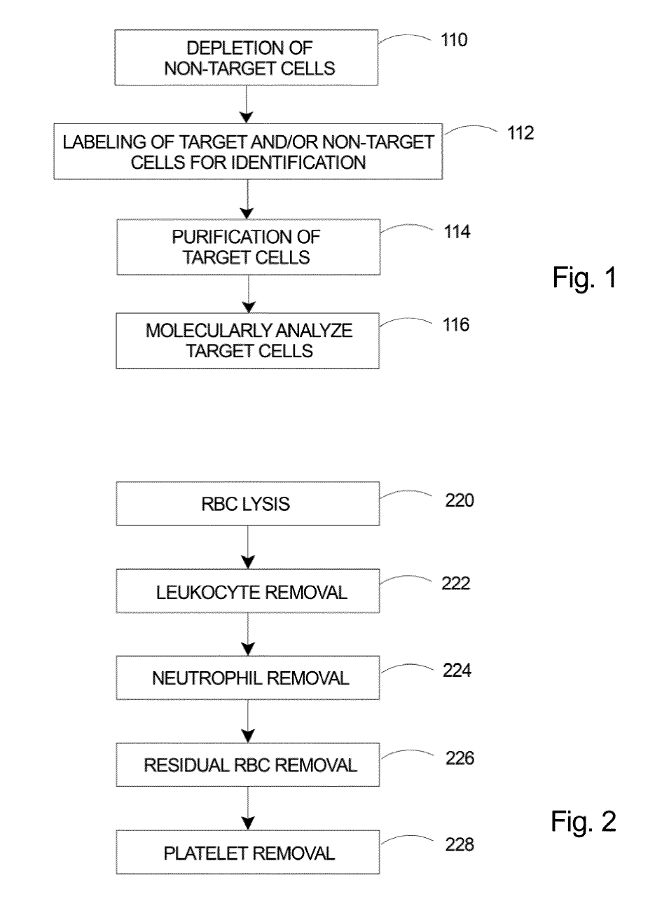

FIG. 1 outlines one embodiment of a method for conducting a molecular analysis on target cells of a complex biological fluid. Reference will now be made in detail to various embodiments of the subject matter, examples of which are illustrated in the accompanying drawings. While the subject matter discussed herein will be described in conjunction with various embodiments, it will be understood that they are not intended to limit the described subject matter to these embodiments. On the contrary, the presented embodiments of the invention are intended to cover alternatives, modifications and equivalents that may be included within the spirit and scope of the various embodiments as defined by the appended claims. Furthermore, in the following description of embodiments, numerous specific details are set forth in order to provide a thorough understanding of embodiments of the subject matter. However, embodiments may be practiced without these specific details. In other instances, well known methods and procedures have not been described in detail as not to unnecessarily obscure aspects of the described embodiments.

At step 110, the complexity of the biological fluid is reduced by at least a partial selective (or differential) depletion of the non-target cells. In this step, in one embodiment, a modest or moderate reduction of the complexity of the biological fluid is desired and achieved. Examples of non-target cells can include, but are not limited to, red blood cells (RBCs) and white blood cells (WBCs). In one embodiment, a technique that is used for differential depletion of non-target cells can include lysis of RBCs using various solutions such as an ammonium chloride solution.

In non-exclusive alternative embodiments, the technique can include RBC removal using filtration or induced paramagnetic separation. RBCs and WBCs can be differentially removed using techniques of microfluidic separation, differential electrophoretic mobility, Magnetic Activated Cell Sorting (MACS), Fluorescence Activated Cell Sorting (FACS), centrifugation, gradient isolation and other suitable techniques. Utilizing the techniques provided herein, the retention of target cells over the depletion of non-target cells such as RBCs and WBCs is increased. In one embodiment, combining this initial step with the downstream steps provides increased speed and target retention, and ultimately, a higher likelihood of achieving or approaching cell purity. In one embodiment, lysis of the RBCs and/or WBCs can reduce the total number of cells from approximately 1×10⁹ cells per ml. to fewer than approximately 1×10⁸ cells per ml. In non-exclusive alternative embodiments, the number of cells can be reduced to fewer than approximately 5×10⁷ cells per ml. or 1×10⁷ cells per ml.

As set forth in greater detail below, concurrently or following removal of at least a portion of the RBCs and/or WBCs, other types of cells can also be removed using various techniques including, but not limited to, magnetic depletion and/or separation. With this technique, in one embodiment, the number of cells can be reduced from approximately 1×10⁹ cells per ml. (or fewer after RBC and/or WBC depletion) to fewer than approximately 1×10⁷ cells per ml. In non-exclusive alternative embodiments, the number of cells can be reduced to fewer than approximately 5×10⁶ cells per ml., 1×10⁶ cells per ml., 5×10⁵ cells per ml., or 1×10⁵ cells per ml. Stated another way, in non-exclusive alternative embodiments, the total cell count of the biological fluid can be reduced by a factor of at least approximately 1×10², 5×10², 1×10³, 5×10³, or 1×10⁴, or even greater.

It should be noted that it is not necessary to remove 100% of the non-target cells or any one type of non-target cell from the biological fluid at this step. In fact, attempting to remove 100% of one or more types of non-target cells from the biological fluid at this step tends to reduce the retention of the target cells. Therefore, it is only necessary to use this first step as a preliminary screening process to remove a certain percentage of the population of non-target cells. In one embodiment, approximately 99.99% of the non-target cells are removed. In other words, the non-target cells are reduced by a factor of approximately 10⁴. In various alternative embodiments, less than approximately 99.9%, 99%, 90%, 80%, 70%, 60%, 50%, 40%, 30%, 20%, 10% or 5% of the non-target cells are removed depending. upon (i) the type of biological fluid being analyzed, (ii) the types of target and non-target cells within the biological fluid, and/or (iii) the desired percentage of purity of the target cells from the biological fluid.

FIG. 2 is a flow chart illustrating one embodiment of a method for reducing complexity of the complex biological fluid by selectively depleting at least a portion of the non-target cells. It is understood that not all of the steps outlined in FIG. 2 are necessary. Further, it is similarly understood that additional steps not illustrated in FIG. 2 can be carried out to selectively deplete other types of cells not described herein.

As provided above, at step 220, the RBCs (and/or WBCs) are selectively depleted by the lysis techniques described herein or by another suitable method that depletes the RBCs and/or WBCs.

At step 222, some or all of the leukocytes of the biological fluid can be removed. In one embodiment, anti-CD45-covered magnetic beads can be added to a suspension of the biological fluid remaining following RBC and/or WBC depletion. The volume of anti-CD45-covered magnetic beads can vary depending upon the total volume of the suspension. For example, in one embodiment, approximately 40 μl of anti-CD45-covered magnetic beads can be added per 1 ml. of suspension. However, it is recognized that more or less than 40 μl of anti-CD45-covered magnetic beads can be added per 1 ml. of suspension. The leukocytes effectively “stick” to the anti-CD45-covered magnetic beads and can consequently be removed from the suspension. Alternatively, another type of antibody can be used in conjunction with magnetic beads to remove the leukocytes.

At step 224, some or all of the neutrophils of the biological fluid can be removed. In one embodiment, anti-CD16-covered magnetic beads can be added to the suspension of biological fluid remaining following RBC and/or WBC depletion. The volume of anti-CD16-covered magnetic beads can vary depending upon the total volume of the suspension. For example, in one embodiment, approximately 200 μl of anti-CD16-covered magnetic beads can be added per 1 ml. of suspension. However, it is recognized that more or less than 200 μl of anti-CD45-covered magnetic beads can be added per 1 ml. of suspension. The neutrophils effectively “stick” to the anti-CD16-covered magnetic beads and can consequently be removed from the suspension. Alternatively, another type of antibody can be used in conjunction with magnetic beads to remove the neutrophils.

At step 226, some or all of the residual RBCs of the biological fluid can be removed. In one embodiment, anti-CD235a-covered magnetic beads can be added to the suspension of biological fluid remaining following the initial lysing of the RBCs and/or WBCs described previously. The volume of anti-CD235a-covered magnetic beads can vary depending upon the total volume of the suspension. The RBCs effectively “stick” to the anti-CD235a-covered magnetic beads and can consequently be removed from the suspension. Alternatively, another type of antibody can be used in conjunction with magnetic beads to remove the RBCs.

At step 228, some or all of the platelets of the biological fluid can be removed. In one embodiment, anti-CD41a-covered magnetic beads can be added to the suspension of biological fluid remaining following RBC and/or WBC depletion. The volume of anti-CD41a-covered magnetic beads can vary depending upon the total volume of the suspension. For example, in one embodiment, approximately 10 μl of anti-CD41a-covered magnetic beads can be added per 1 ml. of suspension. However, it is recognized that more or less than 10 μl of anti-CD41a-covered magnetic beads can be added per 1 ml. of suspension. The platelets effectively “stick” to the anti-CD41a-covered magnetic beads and can consequently be removed from the suspension. Alternatively, another type of antibody can be used in conjunction with magnetic beads to remove the platelets.

It is recognized that in one embodiment, removal of cells that occurs in some or all of steps 222, 224, 226 and/or 228 can occur either simultaneously by using an “antibody cocktail” that includes a plurality of antibody-covered magnetic beads, or the removal can be carried out in a sequence utilizing one or more antibodies at a time, which can occur in a sequence that differs from steps 222, 224, 226 and 228 described above, for example.

Referring back to FIG. 1, at step 112, the target and/or non-target cells are labeled (also referred to as “staining”) for identification. In various embodiments, these labels can include one or more of fluorescent, colorimetric, magnetic and biochemical labels. In one embodiment, labels may bind directly to target cells (DNA, protein, carbohydrates, sugars and other biochemical molecules) or the labels can be tags (i.e. avidin, etc.) on antibodies, nucleic acid probes, other biochemical molecules, or non-target cells. In non-exclusive alternative embodiments, the labels can be used to label some or all cell nuclei, some or all viable cells, only the rare cells, only blood cells, subpopulations of blood and/or rare cells such as those in key biological states of interest including mitosis and/or cells responding to drug treatment.

In one embodiment, a plurality of different antibody specificities can be used which increases the detection of rare cells. For instance, epithelial cells and/or other types of cells such as WBCs can be labeled with phycoerithritin (PE) labeled antibody solutions such as CD326-PE (Epithelial Cell Adhesion Molecule, also referred to as “EpCam”), pan-cytokeratin antibodies, anti-cytokeratin (anti-CK), anti-CD45-TC,2′-(4-Ethoxyphenyl)-5-(4-mrthly-1-piperazinyl)-2,5′-bis-1H enzimidazole.3HCl (also referred to as “H33342”) and/or other suitable labels, as non-exclusive examples. Additionally, or in the alternative, organ-specific (or tissue-specific) markers can be included in the plurality of labels to increase the likelihood that cells from rare tumor types that may have low expression of EpCam or cytokeratin are also detected. For example, tissues such as the pancreas present MUC1, ErbB2 and MUC4 proteins, colorectal cells present CEACAM, prostate cells present PSA, lung cells present EGFR and Ovarian cells present CA-125, CA-72-4, CA-19-9 and CEA, as non-exclusive examples.

The specific volumes of any or all of the above solutions can be varied depending upon the total volume of target and non-target cells being labeled. In some embodiments, a plurality of antibodies can use the same fluorescent label or distinct labels for differentiation as needed. In certain embodiments, a labeling strategy can be advantageous over binding the target cells to beads or surfaces which do not readily have access to binding of cytoplasmic markers and require release strategies that often fragment cells for many downstream analyses.

At step 114, target cells are isolated based on their size and/or label. As used herein, microfluidic sorting can include microfluidic sorting apparatus such as a microfluidic Fluorescence Activated Cell Sorting apparatus (also referred to herein as “μFACS” or “microfluidic FACS”). As used herein, microfluidic FACS is defined as one or more devices which use fluorescence and/or optical detection and measurements to segregate cells based on their size and/or labels. Cells flow through these devices in a relatively narrow fluid stream at relatively low shear stress. Further, these devices can be configured for flow rates that facilitate accurate label identification and subsequent sorting at relatively high efficiency from a relatively small number of cells. These instruments can sort through cells in numbers that range from less than one thousand to greater than one million, which is a departure from traditional FACS instruments. In the present invention, the combination of one or more of step 1 (low purity and modest complexity reduction), step 2 (labeling of target and or non-target cells for identification) and step 3 (μFACS for rapid purity generation of the initially high complexity sample) generates unexpected results of substantial purity, greater target retention and increased speed for utilization in step 116, as explained in greater detail below.

At step 116, the target cells obtained from steps 110, 112 and 114 are molecularly analyzed. The methods provided herein isolate cells that are compatible with numerous downstream molecular analysis applications. These applications can include, as non-exclusive representative examples, DNA analyses, RNA analyses, antigen expression, proteomic analyses and other biological assays as known to one skilled in the art. More specifically, DNA analyses can include DNA microarrays, Comparative Genomic Hybridization (CGH), analysis for rare mutant alleles (single-nucleotide polymorphism (SNP), short tandem repeat (STR), and other microsatellite mutations), mitochondrial DNA, or fluorescent in-situ hybridization (FISH), as non-exclusive examples. RNA analyses can include transcriptional analysis via RT-qPCR and RNA microarrays, as non-exclusive examples. Proteomic analysis can include cell signaling, mass spectrometry, and sub-cellular protein localization, as non-exclusive examples. Biological assays can include viability, drug sensitivity and enzymology, as non-exclusive examples. It is understood that the foregoing listing of various types of molecular analyses are provided as examples only, and are not intended to limit the scope of the present invention in any manner.

Many analyses rely on purity for more accurate results, including but not limited to: CGH, rare allele detection and/or transcriptional profiling. In these cases, enrichment methods are not enabling. For instance, determining the chromosome copy number in aneuploid fetal cells without the presence of contaminating maternal euploid cells is crucial. In all cases, having pure populations can allow more acute and/or accurate measurements of the population of cells and the variability inherent within. This is particularly important for therapeutic applications, for example.

Advantageously, one or more of the methods provided herein do not require harsh fixatives or binding of cells to surfaces such as slides, chips or beads. Binding to surfaces can require manipulation and cell release for some characterizations or for collection and injection back into a host. While manipulating cells adhered to surfaces may not only be harsh, often resulting in a loss of viability or partial destruction of the cell, such manipulations are time consuming and inherently not commercially scalable. However, in an alternative embodiment, cells can be fixed and/or otherwise adhered to slides or other surfaces used by one familiar in the art, if desired.

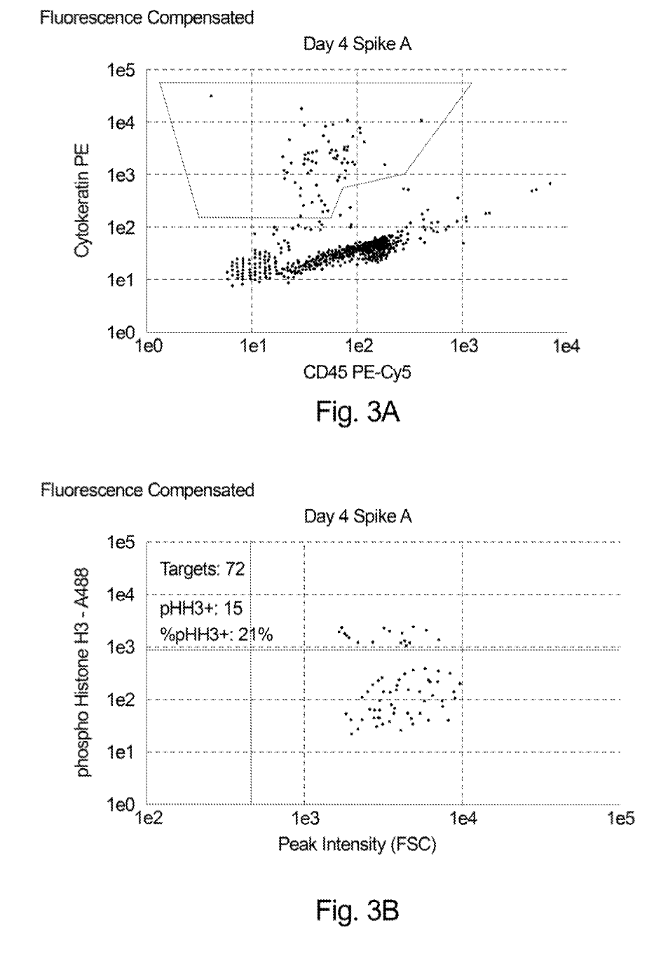

FIGS. 3A and 3B are graphs illustrating one embodiment of a method for conducting a molecular analysis on target cells of a complex biological fluid that includes target cells and non-target cells. In this embodiment, the method includes selective depletion of non-target cells with anti-CD45 and anti-CD16 (step 110 in FIG. 1), labeling the target cells with CD45, pan CK, nuclear stain and phosphorylated histone H3 (step 112 in FIG. 1), isolating the target cells using microfluidic FACS (step 114 in FIG. 1), and molecularly analyzing the target cells for levels of phosphorylated histone H3, which is a marker that indicates mitotic arrest.

In this example, 100 prostate cancer cells treated with taxol to induce mitotic arrest were spiked-in to whole blood and stored for four days. Using the methods provided herein, 72 target cells were recovered (illustrated in the somewhat trapezoidal region in the upper portion of the graph in FIG. 3A), thereby providing a recovery rate of 72%. No non-target cells were observed in this trapezoidal region. The remaining cells in the lower portion of FIG. 3A represent non-target WBCs. In FIG. 3B, the target cells were efficiently isolated and the response of the target cells to the phosphorylated histone H3 was accurately determined via molecular analysis. As illustrated in FIG. 3B, the cells above the horizontal line at phosphorylated histone H3 level of 1×10³ were indicative of having responded to treatment.

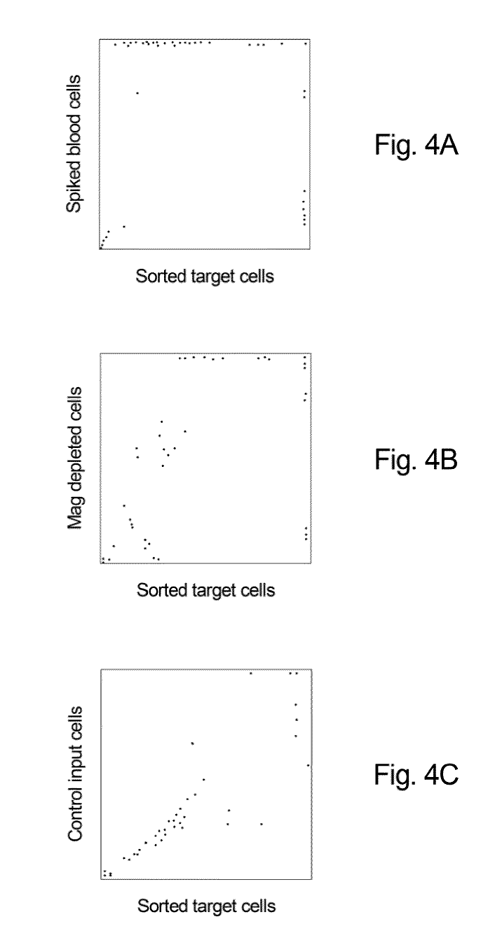

FIGS. 4A-4C illustrates actual results of the purification of target cells from blood by analyzing cell transcripts. More specifically, FIG. 4A is a graph of an analysis of cell transcripts from human blood that includes approximately 50 spiked-in target cells, as a comparison to the sorted target cells; FIG. 4B is a graph of an analysis of cell transcripts of blood cells in the biological fluid described relative to FIG. 4A following magnetic depletion, as a comparison to the sorted target cells; and FIG. 4C is a graph of an analysis of cell transcripts of blood cells in the biological fluid described relative to FIG. 4B following sorting by a microfluidic Fluorescence Activated Cell Sorting apparatus, as a comparison to the sorted target cells.

The purification of target cells from blood can be verified by analysis of cell transcripts. If transcripts are measured by RT-qPCR the resulting Ct values can be compared for each transcript from one sample to another. When Cts from target cells are compared to the whole blood the cells were spiked into, there is virtually no correlation, as illustrated in FIG. 4A. The correlation is also poor when the comparison is the target cells compared to the WBC depleted blood, as illustrated in FIG. 4B. However, comparison of the sorted target cells to the original spiked-in cells results in a substantially diagonal line representing approximately a 1:1 correlation or similarity of transcript levels, as illustrated in FIG. 4C. Thus the input cells have a near identical transcriptional profile to those that were purified out of the spike-in blood. The present invention cannot only generate substantially purified samples, it enables transcriptional analysis of rare cells.

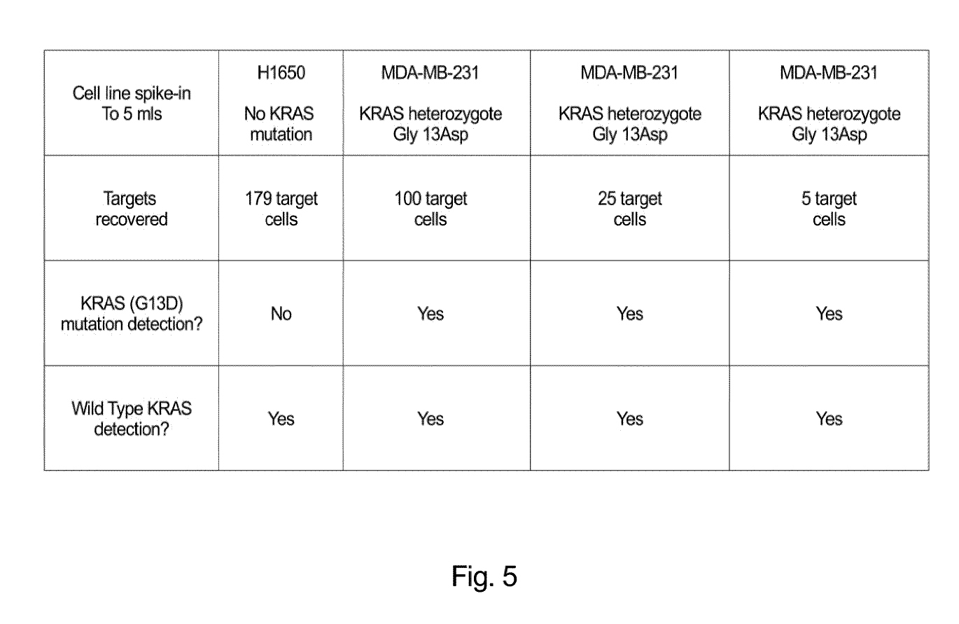

FIG. 5 is a table illustrating how molecular analysis including mutation detection is enabled by the methods that follow the steps of the present invention. In this embodiment, various target cells, including differing types and amounts, were spiked into blood in four separate experiments, shown in the second, third, fourth and fifth columns in FIG. 5. It is well known to those skilled in the art that in order for mutation detection to occur with any degree of certainty, at least approximately 1% to 10% purity must be present in the sample. The second column of the table in FIG. 4 illustrates that as a control, a cell line of 179 target cells of H1650, having no KRAS mutation, were spiked into 5 mls of blood. Following utilization of the methods provided herein, as expected, no KRAS (G13D) mutation was detected, however, the wild type allele was detected.

The third column in FIG. 5 illustrates that a 100 target cells of cell line MDA-MB-231 was spiked into 5 mls of blood. Following utilization of the methods provided herein, the KRAS (G13D) mutation was detected, as was the wild type allele. The detection of both the KRAS mutant allele and the wild type allele is indicative that the recovery of the target cells was sufficient to yield at least 1% to 10% purity of the target cells.

The fourth column in FIG. 5 illustrates that 25 target cells of cell line MDA-MB-231 was spiked into 5 mls of blood. Following utilization of the methods provided herein, the KRAS (G13D) mutation was detected, as was the wild type allele. The detection of both the KRAS mutant allele and the wild type allele is indicative that the recovery of the target cells was sufficient to yield at least 1% to 10% purity of the target cells.

The fifth column in FIG. 5 illustrates that 5 target cells of cell line MDA-MB-231 was spiked into 5 mls of blood. Following utilization of the methods provided herein, the KRAS (G13D) mutation was detected, as was the wild type allele. The detection of both the KRAS mutant allele and the wild type allele is indicative that the recovery of the target cells was sufficient to yield at least 1% to 10% purity of the target cells.

Cells of diagnostic and therapeutic value exist in body fluids and tissue suspensions. Example fluids can include, but are not limited to, circulatory blood, disassociated tissue suspensions, cord blood, urine, sputum, cerebral spinal fluid, fecal matter suspensions and peritoneal fluid, which have all been monitored for biomarkers of disease, or other suitable fluids. Target cells of interest can include, but are not limited to, circulating epithelial cells such as those indicative of cancer, and circulating endothelial cells as markers of vascular damage such as myocardial infarction, vasculitis and transplantation. For example, collection of fetal cells from maternal blood or cord blood would enable a non-invasive prenatal health diagnosis to be made. Stem cells may also be isolated from a patient's blood, marrow, organs or other body fluids, appropriately differentiated, propagated and used to supplant failing tissues or organs. Other target cells can include one or more of tumor cells, endothelial cells, myocardial cells and lymphocytes, as non-exclusive examples.

One or more of the methods provided herein have essentially no theoretical volume limit and no measured complexity limit. Rare cell events may be present at less than 1 cell/mL of blood. Further, one or more of the methods provided herein overcome the previously unsolved problem regarding scalability for relatively large initial fluid volumes, such as greater than 7.5 mLs, for instance. Biological fluids have varying degrees of complexity, with blood having the highest density of cells (1×10⁹/mL) in human body fluids. In certain embodiments, one or more of the methods provided herein can rapidly reduce approximately 100 mLs of blood (approximately 1×10¹¹ cells) to substantially purified populations of cells on the order of μl volumes. Thus, input and output volumes of several orders of magnitude can be managed and achieved utilizing the methods provided herein. In non-exclusive alternative embodiments, the target cells can be recovered in a volume of at least approximately 5 μl and less than approximately 1 ml, or at least approximately 10 μl and less than approximately 0.1 ml. Still alternatively, other blood volumes can be rapidly reduced to substantially purified populations of cells on the order of volumes that are somewhat proportionate to the above examples.

Many of the methods provided herein allow for relatively high purity and recovery retention rates. Previously, retention of target cells was measureable when a gold standard exists for the quantity existing in the sample. The best way to generate such a standard is to spike-in a known quantity of cells and measure the output purity and recovery retention rates. The present invention is capable of routinely achieving greater than approximately 60%, 70%, 80% or 90% purity and/or recovery retention.

Additionally, one or more of the methods described herein can permit a decreased processing time. Further, the entire process can be scalable by a single operator in a single location. This has commercial value in the ability to process many samples simultaneously and provide data back to a clinician, researcher or patient in point of care settings. Additionally, many of the processes disclosed herein allow the identification of many transient biological events such as cell signaling and would be missed by longer enrichment methodologies.

In certain embodiments, selective depletion of non-target cells and sorting of target cells can occur by a multi-parameter analysis. Size and labels such as DNA content and as many external or cytoplasmic antigens as one wishes can be used individually or in sets of positive and negative labels for selection. Conventional techniques that rely on affinity capture are not easily scaled to use multiple antibodies due to the loss of co-operativity that results as a single capture antibody is diluted out by second, third or other additional antibody types. This issue is negated when antibody labels are bound to cells in bulk solution. Information on the size of the target cells can also be collected using the methods of the present invention and can be incorporated into the selection process, if desired.

In one embodiment, the duration of the entire method described herein can be less than approximately 3 hours. In non-exclusive alternative embodiments, the duration of the entire method described herein can be less than approximately 6 hours or less than approximately one day.

While the methods as shown and disclosed herein are fully capable of obtaining the objects and providing the advantages herein before stated, it is to be understood that they are merely illustrative of the presently preferred embodiments of the invention and that no limitations are intended to the details of the methods, processes, construction or design herein shown and described.

What is claimed is:

1. A method for conducting a molecular analysis on target cells of a complex biological fluid that includes target cells and non-target cells, the method comprising the steps of:
reducing the complexity of the biological fluid by selectively depleting at least a portion of the non-target cells from the biological fluid;
labeling the target cells or the non-target cells for identification;
isolating the target cells from the non-target cells based on one of (i) a size, or (ii) a label, of the target cells or the non-target cells using a microfluidic sorting apparatus; and
molecularly analyzing the target cells.

2. The method of claim 1 wherein the step of reducing includes separating the non-target cells from the biological fluid using a Magnetic Activated Cell Sorting apparatus.

3. The method of claim 2 wherein the step of separating includes using magnetic beads that include a plurality of different antibodies.

4. The method of claim 1 wherein the step of reducing includes lysing at least some of the non-target cells.

5. The method of claim 4 wherein the step of lysing includes using an ammonium chloride solution.

6. The method of claim 1 wherein the step of reducing includes filtration of at least some of the non-target cells.

7. The method of claim 6 wherein the antibodies include one or more of CD45, CD16 and CD235a

8. The method of claim 1 wherein the step of labeling includes labeling both the target cells and the non-target cells.

9. The method of claim 1 wherein the step of labeling includes using a label selected from the group consisting of fluorescent, colorimetric, magnetic and biochemical labels.

10. The method of claim 1 wherein the step of labeling includes labeling at least some of the target cells with organ-specific markers.

11. The method of claim 1 wherein the microfluidic sorting apparatus includes a microfluidic Fluorescence Activated Cell Sorting apparatus.

12. The method of claim 1 wherein the step of isolating includes purifying the target cells based on a label of the target cells and the non-target cells.

13. The method of claim 1 wherein the step of isolating includes purifying the target cells based on the size and the label of either the target cells or the non-target cells.

14. The method of claim 1 wherein the number of target and non-target cells in the biological fluid is less than approximately 1×10¹¹ cells in a fluid volume of less than approximately 100 mls.

15. The method of claim 14 wherein the number of target and non-target cells in the biological fluid is greater than approximately 1×10⁸ in a fluid volume of greater than approximately 0.1 mls.

16. The method of claim 1 wherein the step of isolating includes recovering the target cells in a volume of at least approximately 5 μl and less than approximately 1 ml.

17. The method of claim 1 wherein the step of isolating includes recovering the target cells in a volume of at least approximately 10 μl and less than approximately 0.1 ml.

18. The method of claim 1 wherein, the biological fluid is selected from the group consisting of blood sputum, peritoneal fluid, tissue cell suspension, fine needle aspirates, fecal matter and cerebral spinal fluid.

19. The method of claim 1 wherein the step of isolating yields a purity of at least approximately 60%.

20. The method of claim 1 wherein the target cells are selected from the group consisting of tumor cells, fetal cells, stem cells, endothelial cells, myocardial cells and lymphocytes.

21. The method of claim 1 wherein the duration of the method is less than approximately 6 hours.

22. The method of claim 1 wherein the step of isolating includes retention of the target cells being at least 80%.

23. The method of claim 1 wherein the step of molecularly analyzing the target cells includes one of RNA analysis, antigen expression and mutation profiling.

24. A method for conducting a molecular analysis on target cells of a complex biological fluid that includes target cells and non-target cells, the method comprising the steps of:
reducing the complexity of the biological fluid by selectively depleting at least a portion of the non-target cells from the biological fluid, the step of selectively depleting including (i) lysing at least some of the non-target cells, and (ii) paramagnetically separating the non-target cells from the target cells;
labeling the target cells or the non-target cells for identification;
isolating the target cells from the non-target cells based on one of (i) a size, or (ii) a label, of the target cells or the non-target cells using a microfluidic Fluorescence Activated Cell Sorting apparatus; and
molecularly analyzing the target cells.

25. The method of claim 24 wherein the step of labeling includes using a label selected from the group consisting of fluorescent, colorimetric, magnetic and biochemical labels.

26. The method of claim 24 wherein the step of labeling includes labeling at least some of the target cells with organ-specific markers.

27. The method of claim 24 wherein the step of isolating includes isolating the target cells based on a label of the target cells and the non-target cells.

28. The method of claim 24 wherein the step of isolating includes isolating the target cells based on the size and the label of either the target cells or the non-target cells.

29. The method of claim 24 wherein the number of target and non-target cells in the biological fluid is greater than approximately 1×10⁸ in a fluid volume of greater than approximately 0.1 mls, and less than approximately 1×10¹¹ cells in a fluid volume of less than approximately 100 mls.

30. The method of claim 24 wherein the step of isolating includes recovering the target cells in a volume of at least approximately 5 μl and less than approximately 1 ml.

31. The method of claim 24 wherein the biological fluid is selected from the group consisting of blood sputum, peritoneal fluid, tissue cell suspension, fine needle aspirates, fecal matter and cerebral spinal fluid.

32. The method of claim 24 wherein the step of isolating yields a purity of at least approximately 70%.

33. The method of claim 24 wherein the target cells are selected from the group consisting of tumor cells, fetal cells, stem cells, endothelial cells, myocardial cells and lymphocytes.

34. The method of claim 24 wherein the step of isolating includes retention of the target cells being at least 80%.

35. The method of claim 24 wherein the step of molecularly analyzing the target cells includes one of RNA analysis and mutation profiling.

36. A method for conducting a molecular analysis on target cells of a complex biological fluid that includes target cells and non-target cells, the method comprising the steps of:
reducing the complexity of the biological fluid by selectively depleting at least a portion of the non-target cells from the biological fluid, the step of selectively depleting including (i) lysing at least some of the non-target cells, and (ii) paramagnetically separating the non-target cells from the target cells;
labeling the target cells for identification;
isolating the target cells from the non-target cells based on one of (i) a size, or (ii) a label, of the target cells or the non-target cells using a microfluidic Fluorescence Activated Cell Sorting apparatus to yield a purity of at least approximately 70% and a recovery retention of at least 70%; and
molecularly analyzing the target cells using one of mutation profiling or an RNA analysis.
